(19) United States
(12) Patent Application Publication
(10) Pub. No.: US 2007/0065898 A1
(43) Pub. Date: Mar. 22, 2007

(54) Fluorescence polarization assays for binding of caspase inhibitors and probes therefor

(75) Inventors: Paul Dollings, Newtown, PA (US); Gregory Tawa, Doylestown, PA (US); Ann Aulabaugh, Upper Saddle River, NJ (US)

Correspondence Address: COZEN O' CONNOR, P. C., 1900 MARKET STREET, PHILADELPHIA, PA, 19103-3508, US

(73) Assignee: Wyeth, Madison, NJ (US)

(21) Appl. No.: 11/524,700

(22) Filed: Sep. 21, 2006

Related U.S. Application Data
(60) Provisional application No. 60/719,253, filed on Sep. 21, 2005.

(51) Int. Cl. C12Q 1/37; C07D 405/14

(52) U.S. Cl. 435/23; 548/409; 548/454

(57) ABSTRACT

This invention provides a fluorescence polarization assay useful in the detection and evaluation of caspases inhibitors. This invention also provides novel fluorescent probes used in the fluorescence polarization assay, and methods for making the fluorescent probes, which have the Formula I: 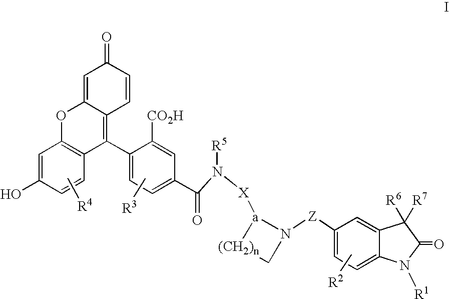 wherein the constituent variables are as defined herein.

This application claims benefit of priority to U.S. provisional patent application Ser. No. 60/719,253 filed on Sep. 21, 2005, which is hereby incorporated in its entirety.

### FIELD OF THE INVENTION

The present invention relates to fluorescence polarization assays useful in the detection and evaluation of potential caspase inhibitors. The present invention also relates to novel fluorescent probes used in the fluorescence polarization assay, and methods of making such fluorescent probes.

### BACKGROUND OF THE INVENTION

Cell death is generally classified into one of two forms, necrosis or apoptosis. Necrosis typically occurs in response to severe physiological or environmental insult. Cells dying by necrosis show a distinct pattern of cellular breakdown, which eventually results in cell autolysis. The resulting release of cellular contents can cause inflammation in the surrounding tissues, furthering cell injury. Apoptosis on the other hand is a controlled or programmed series of cellular events ultimately leading to cell death. It is a mechanism for an organism to remove unwanted cells and is an important part of normal physiology. The two most often cited examples of apoptosis are in fetal development and in immune cell development. However, excessive or insufficient apoptosis can play a role in disease. Diseases in which there is an excessive accumulation of cells and insufficient apoptosis include cancer, inflammatory disorders and autoimmune diseases. Disorders in which excessive apoptotic cell death has been observed include neurodegenerative conditions such as Alzheimer's and Parkinson's diseases and ischemic stroke, brain or myocardial diseases. Tissue damage following stroke or myocardial infarction is largely apoptotic and there is growing evidence that the inhibition of apoptosis following ischemic injury can lessen the tissue damage.

One of the most specific molecular markers for apoptosis is the activation of the family of cysteine-dependent aspartate proteases, which are known as caspases. At least 11 human caspases have been characterized and these can be subdivided into three groups based on homology and substrate specificity. Group I caspases including 1, 4, 5, appear to be predominately involved in inflammation. Group II caspases including 6, 8, 9 and 10, are initiators of apoptotic signaling and further caspase activation. Group III caspases, including 3 and 7, are predominantly effector enzymes responsible for degrading cellular substrates in a highly specific manner. Although the precise repertoire of caspases involved in ischemic neuronal cell death have not been fully elucidated, histochemical and biochemical data combine to show the presence of activated caspases from each class in adult ischemic brain (Chen et al., J. Neuroscience, 18: 4914-4928 (1998), Krupinski et al., Neurobiol. Dis. 7: 332-342 (2000), Benchoua et al., J. Neuroscience 21: 7127-7134 (2001), Namura et al., J. Neuroscience. 18: 3659-3668 (1998)).

The utility of a caspase inhibitor for the treatment of a number of mammalian diseases associated with an increase in cellular apoptosis has been demonstrated using peptidic caspase inhibitors. For example, in rodent models caspase inhibitors have been shown to reduce infart size and inhibit cardiomyocyte apoptosis after myocardial infarction, to reduce brain lesion volume and neurological deficit resulting from stroke, to reduce post traumatic apoptosis and neurological deficit in traumatic brain injury, to be effective in treating fulminant liver destruction and to improve survival after endotoxic shock. (Yaoita, et al., Circulation, 97: 276 (1998), Endres et al., J. Cerebral Blood Flow and Metabolism, 18: 238 (1998), Cheng, et al., J. Clin. Invest., 101: 1992 (1998); Yakovlev, et al., J. Neuroscience, 17: 7415 (1997), Rodriquez, et al., J. Exp. Med., 184: 2067 (1996), Grobmyer, et al., Mol. Med., 5: 585 (1999)).

Caspase inhibitors may be useful for the treatment of osteoarthritis. A recent study demonstrated an increase in the level of the active form of caspase 3 in osteoarthritis chondrocytes. (Pelletier, et al., Arthritis & Rheumatism, 43(6):1290 (2000)). In osteoarthritis chondrocytes, the distribution of cells staining for caspase 3 was superimposable to that of cells undergoing apoptosis. The strong correlation between caspase 3 and apoptosis supports the notion that caspase 3 plays a role in chondrocyte apoptosis.

Fluorescence polarization assay techniques are based on the principle that a fluorescence labeled compound will emit fluorescence when excited by plane polarized light, having a degree of polarization inversely related to its rate of rotation. If the labeled molecule remains stationary throughout the excited state it will emit light in the same polarized plane; if it rotates while excited, the light emitted is in a different plane. Specifically, when a large labeled molecule is excited by plane polarized light, the emitted light remains highly polarized because the fluorophore is constrained (by its size) from rotating between light absorption and fluorescent light emission. When a smaller molecule is excited by plane polarized light, its rotation is much faster than the large molecule and the emitted light is more depolarized. Polarization is related to the time it takes a fluorescence labeled molecule to rotate through an angle of approximately 68.5 degrees: designated the correlation time. Correlation time is related to viscosity, absolute temperature and molecular volume. If viscosity and temperature are held constant, correlation time, and therefore, polarization, are directly proportional to the molecular volume. Changes in molecular volume may be due to molecular binding, dissociation, synthesis, degradation, or conformational changes of the fluorescence labeled molecule. Accordingly, when plane polarized light is passed through a solution containing a relatively high molecular weight fluorescence labeled compound, the degree of polarization of the emitted light will, in general, be greater than when plane polarized light is passed through a solution containing a relatively low molecular weight fluorescence labeled compound.

The term “Fluorescence polarization” (P) is defined as:

$$P = \frac{Parallel Intensity - Perpendicular Intensity}{Parallel Intensity + Perpendicular Intensity} \qquad \text{Eq. 1}$$

Parallel Intensity is the intensity of the emission light parallel to the excitation light plane and Perpendicular Intensity is the intensity of the emission light perpendicular to the excitation light plane. Since P is a ratio of light intensities, it is a dimensionless number and has a maximum value of 0.5 for fluorescein.

Fluorescence anisotropy (A) is another term commonly used to describe this phenomenon. It is related fluorescence polarization according to the following formula:

$$A = \frac{2 P}{3 - P} , \qquad \text{Eq. 2}$$

wherein P is fluorescence polarization.

Fluorescence polarization assays are homogeneous in that they do not require a separation step such as centrifugation, filtration, chromatography, precipitation or electrophoresis. Assays can be performed in real time, directly in solution and do not require an immobilized phase. For example, fluorescence polarization has been used to measure enzymatic cleavage of large fluorescein labeled polymers by proteases, DNases and RNases.

It can be seen that fluorescence polarization assays for determining the binding of compounds to caspases can be of great value in the discovery of potential caspase inhibitors. This invention is directed to these, as well as other, important ends.

### SUMMARY OF THE INVENTION

In one aspect, the present invention provides fluorescent polarization assays for the detection and evaluation of potential caspase inhibitors. The assays of the present invention utilize the measurement of fluorescence polarization of a fluorescence-emitting compound that binds to an active site of a caspase.

The present invention is also directed to methods for measuring competitive binding activity of molecules to a caspase. In some embodiments, the methods include combining a fluorescence-emitting compound that binds to the active site of the caspase in a solution containing the caspase, and measurement of the fluorescence polarization of the solution. The solution is then incubated with at least one molecule that may compete with the compound for binding to the caspase, and the fluorescence polarization is then measured. Comparing the fluorescence polarization measurements is then used to quantify any competitive interaction.

In some embodiments, the fluorescence polarization assays of the present invention use novel fluorescent probes that bind to the active sites of caspases. The fluorescent probes, which constitute an aspect of the present invention, have the Formula I: 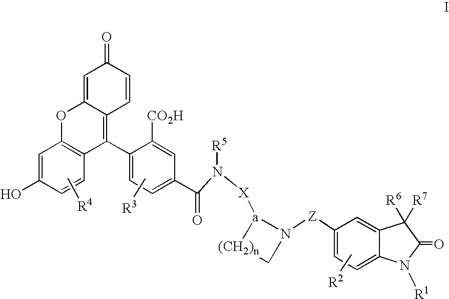
or a salt thereof; wherein:

R¹ is H, C₁-C₆ alkyl or C₇-C₂₄ aralkyl;

R², R³ and R⁴ are, each independently, H, halogen or C₁-C₆ alkyl

R⁵ is H or C₁-C₆ alkyl;

n is 1, 2 or 3;

X is (CH₂)ₘCONR^{5a}—(CH₂)_{q}—, —(CH₂)_{q}— or —(CH₂)ₘSO₂NR^{5a}—(CH₂)_{q}—;

R⁵ is H or C₇-C₆ alkyl;

m is 1, 2, 3, 4, 5 or 6;

q is 0, 1, 2 or 3;

R⁶ and R⁷ are each independently OH or —O—(C₁-C₆ alkyl);
or R⁶ and R⁷ taken together can form ═O or —O—(CH₂)ᵣ—O—, where r is 2 or 3; and Z is CO or SO₂; and

a is a carbon atom, preferably having the “S” configuration.

The present invention also provides synthetic methods for the preparation of the compounds of Formula I, and intermediates useful for preparing the compounds of Formula I.

### DETAILED DESCRIPTION OF THE INVENTION

The present invention is directed to fluorescent polarization assays for the detection and evaluation of potential caspase inhibitors. The present invention is also directed to the use of novel fluorescent probes that bind to the active sites of caspases. The fluorescent probe which constitutes one aspect of the present invention, is a compound having the Formula I: 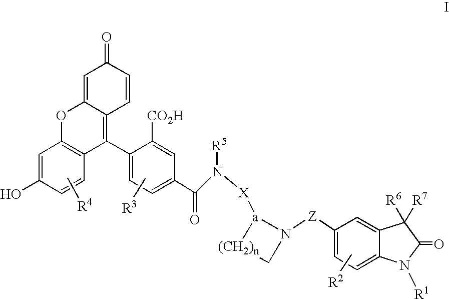
or a salt thereof; wherein:

R¹ is H, C₁-C₆ alkyl or C₇-C₂₄ aralkyl;

R², R³ and R⁴ are, each independently, H, halogen or C₁-C₆ alkyl;

R⁵ is H or C₁-C₆ alkyl;

n is 1, 2 or 3;

X is —(C H₂)ₘCONR^{5a}—(CH₂)_{q}—, —(CH₂)_{q}— or —(CH₂)ₘSO₂NR^{5a}—(CH₂)_{q}—;

R^{5a} is H or C₁-C₆ alkyl;

m is 1, 2, 3, 4, 5 or 6;

q is 0, 1, 2 or 3;

R⁶ and R⁷ are each independently OH or —O—(C₁-C₆ alkyl);
or R⁶ and R⁷ taken together can form ═O or —O—(CH₂)ᵣ—O—, where r is 2 or 3; and

Z is CO or SO₂; and

a is a carbon atom.

In some embodiments, the carbon atom designated “a” in Formula I has the S configuration. In some further embodiments, n is 2. In some further embodiments, X is —CH₂—. In some further embodiments, X is —(CH₂)ₘCONR^{5a}(CH₂)_{q}. In some further embodiments, X is —(CH₂)ₘCONR^{5a}(CH₂)_{q}—, wherein m is 5 and q is 1. In some further embodiments, X is —(CH₂)ₘCONR^{5a}(CH₂)_{q}—, wherein m is 5, q is 1, and R^{5a} is hydrogen.

In some further embodiments, Z is SO₂. In some embodiments, R², R³, R⁴, R⁵ and R^{5a} are each H. In some embodiments, R¹ is methyl.

In some further embodiments, R⁶ and R⁷ taken together form ═O.

In some preferred embodiments, the invention provides a fluorescence probe of Formula I wherein n is 2; X is —CH₂—; Z is SO₂; R², R³, R⁴ and R⁵ are each hydrogen; R⁶ and R⁷ are taken together to form ═O; R¹ is methyl, and the carbon atom designated “a” has the S configuration; i.e., a compound of the formula:

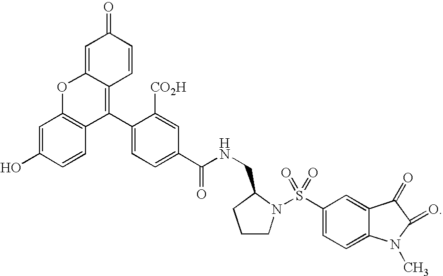

In some preferred embodiments, the invention provides a fluorescence probe of Formula I wherein n is 2; X is —(CH₂)ₘCONR^{5a}(CH₂)_{q}, wherein m is 5, q is 1, and R^{5a} is hydrogen; Z is SO₂; R², R³, R⁴ and R⁵ are each hydrogen; R⁶ and R⁷ are taken together to form ═O; R¹ is methyl, and the carbon atom designated “a” has the S configuration; i.e., a compound of the formula:

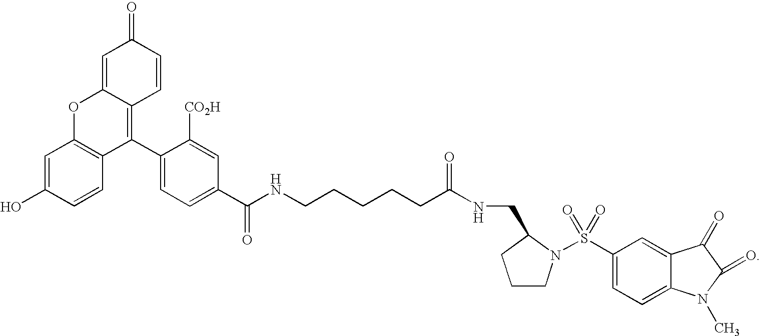

In some embodiments, the present invention provides methods for the preparation of compounds of the invention, having the Formula I above. In some embodiments, the methods include:

a) providing a compound of Formula II: 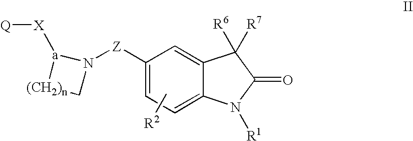
or a salt thereof, wherein:

X is —(CH₂)_{q}—;

R⁶ and R⁷ taken together form —O—(CH₂)ᵣ—O—, where r is 2 or 3; and

Q is NH₂; and

b) reacting the compound of Formula II with a compound of Formula II: 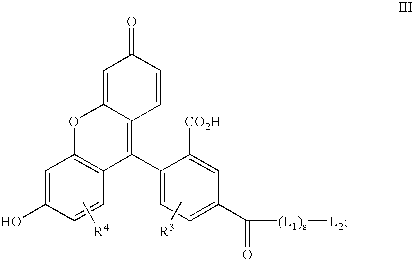
or a salt thereof, wherein:

L₁ is a linker group;

s is 0 or 1; and

L₂ is a leaving group;

for a time and under conditions effective to form the compound of Formula I.

In some embodiments, the carbon atom designated “a” has the S configuration. In some embodiments, L₁ has the formula —NR⁵—(CH₂)ₘCO—, —NR⁵—CH₂)ₘSO₂— or —NH—(CH₂)ₘ—C(═O)—. In some embodiments, L₂ has the formula:

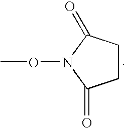

In some further embodiments, L₁ has the formula —NH—(CH₂)ₘ—C(═O)—; and L₂ has the formula:

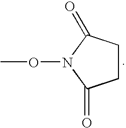

In some further embodiments, m is 5.

In some embodiments, s is 0. In some further embodiments, s is 0 and L₂ has the formula:

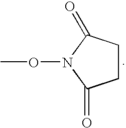

In some embodiments, the atom designated “a” has the S configuration; n is 2; X is —CH₂—; Z is SO₂; R², R³, R⁴ and R⁵ are each hydrogen; R⁶ and R⁷ are taken together to form —O—(CH₂)₃—O—; R¹ is methyl; s is 0; and L₂ has the formula:

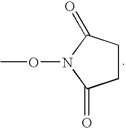

In some embodiments, the atom designated “a” has the S configuration; n is 2; X is —(CH₂)ₘCONR^{5a}(CH₂)_{q}—, wherein m is 5, q is 1, and R^{5a} is hydrogen; Z is SO₂; R², R³, R⁴ and R⁵ are each hydrogen; R⁵ and R⁷ taken together form —O—(CH₂)₃—O—; R¹ is methyl; s is 1; L₁ has the formula —NH—(CH₂)ₘ—C(═O)—; and L₂ has the formula:

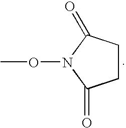

In some embodiments, the methods further include reacting the compound of Formula I wherein R⁶ and R⁷ taken together form —O—(CH₂)ᵣO—, where r is 2 or 3, with an acid to form a compound of Formula I wherein R⁶ and R⁷ taken together form ═O.

In some embodiments, the invention further provides products of the methods described herein.

In the Formulae above, L₁ is an optional linker group. The linker group L₁ can be any of a wide variety of linking groups known in the art. Generally, the linking groups contain a spacer region, and functional groups at the ends of the spacer region that can form attachments with the moieties that are linked together. Spacer regions can contain any of a wide variety of moieties that serve to provide distance between the fluorescent carboxyfluorescein derivative and the enzyme binding moiety. Representative spacers include alkyl groups, for example C₁₋₆ alkyl groups; cycloalkyl groups, preferably C₃₋₆ cycloalkyl groups, including cyclopropyl, cyclobutyl, cyclopentyl and cyclohexyl groups; and C₂₋₆ unsaturated hydrocarbon groups that can contain one or more carbon-carbon double bonds (alkenyl chains), one or more carbon-carbon triple bonds (alkynyl chains), or both one or more carbon-carbon double bond and one or more carbon-carbon triple bond. Further examples of suitable spacers includes polyether groups. Still further suitable spacers include bivalent groups of the formula -Z₁-Z₂-(Z₃)ₛ-, where s is 0 or 1, and each of Z₁, Z₂, and Z₃ is independently selected from the alkyl groups, cycloalkyl groups, unsaturated groups, and polyether groups described above.

Representative functional groups for the ends of the spacer region include activated or protected carboxyl, sulfonyl or amino groups. Such groups can be utilized to form carboxamide or sulfonamide linkages at the ends of the linking group. Thus, non-limiting examples of linking groups for L₁ include those having the formula —NR⁵—(CH₂)ₘCO— or —NR⁵—(CH₂)ₘSO₂—, wherein m is 1, 2, 3, 4, 5 or 6; and wherein R⁵ is H or C₁-C₆ alkyl. In some embodiments, the linking groups for L₁ include those having the formula —NH—(CH₂)ₘ—C(═O)—, where m is an integer of 1 to 6.

In the Formulae above, L₂ is a leaving group. The leaving group is chosen such that it can be displaced by the amino group of the compound of Formula II to form a linkage between the carbonyl group of the fluorescein derivative, or, if present, the reactive functionality of linking group L₁. Many such leaving groups are known to those of skill in the art. In one preferred embodiment, the leaving group is the portion of an N-succinimidyl ester having the formula:

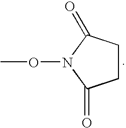

In one aspect, the invention provides intermediates in the preparation of the compounds of Formula I, and methods for their preparation. In some such embodiments, the invention provides method for the preparation of compound of Formula IV: 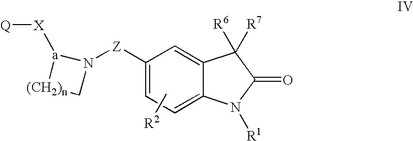
wherein:
R¹ is C₁-C₆ alkyl or C₇-C₂₄ aralkyl;
R² is H, halogen or C₁-C₆ alkyl;
n is 1, 2 or 3;
X is —(CH₂)_{q}—;
q is 0, 1, 2 or 3;
R⁶ and R⁷ taken together form —O—(CH₂)ᵣ—O—, where r is 2 or 3;
Z is CO or SO₂;
Q is NH₂; and
a is a carbon atom;
comprising:

a) providing a compound of Formula V: 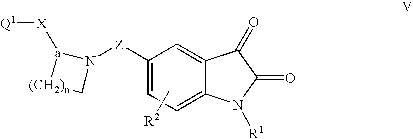
wherein:
R¹ is H;
Q¹ is N(R¹⁰)(R¹¹); and
R¹⁰ is a protecting group and R¹¹ is H;

or R¹⁰ and R¹¹ taken together with the nitrogen atom to which they are attached can form an N-phthalimidyl group;

b) reacting the compound of Formula V with a reagent of formula HO—(CH₂)ᵣ—OH where r is 2 or 3, under conditions effective to form a compound of Formula VI:

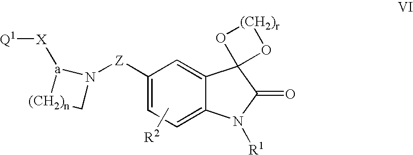

c) reacting the compound of Formula VI from step b) with an alkyl iodide or aralkyl iodide to form a compound of Formula VI wherein R¹ is alkyl or aralkyl; and

d) treating the compound of Formula VI wherein R¹ is alkyl or aralkyl with a reagent effective to remove:

i) the protecting group from R¹⁰; or

ii) the N-phthalimidyl group from N(R¹⁰)(R¹¹); to form the compound of Formula IV.

In some embodiments, the carbon atom designated “a” has the S configuration. In some further embodiments, r is 3. In some further embodiments, n is 2. In some further embodiments, R¹⁰ and R¹¹ taken together with the nitrogen atom to which they are attached form an N-phthalimidyl group. In some further embodiments, R² is H.

In some embodiments, the compound of Formula VI from step b) is reacted with an alkyl iodide to form a compound of Formula VI wherein R¹ is alkyl in step c). In some embodiments, the alkyl iodide is methyl iodide.

In some embodiments, r is 3; n is 2; R¹⁰ and R¹¹ taken together with the nitrogen atom to which they are attached form an N-phthalimidyl group; R² is H; and the compound of Formula VI from step b) is reacted with methyl iodide to form a compound of Formula VI wherein R¹ is methyl in step c).

As used herein, the term “alkyl” is meant to refer to a monovalent or divalent saturated hydrocarbon group which is straight-chained or branched. Example alkyl groups include methyl (Me), ethyl (Et), propyl (e.g., n-propyl and isopropyl), butyl (e.g., n-butyl, isobutyl, s-butyl, t-butyl), pentyl (e.g., n-pentyl, isopentyl, neopentyl) and the like. An alkyl group can contain from 1 to about 20, from 2 to about 20, from 1 to about 10, from 1 to about 8, from 1 to about 6, from 1 to about 4, or from 1 to about 3 carbon atoms. As used herein, the term “lower alkyl” is intended to mean alkyl groups having up to six carbon atoms.

The carbon number, as used in the definitions herein, refers to carbon backbone and carbon branching, but does not include carbon atoms of substituents, such as alkoxy substitutions and the like.

As used herein, “aryl” refers to aromatic carbocyclic groups including monocyclic or polycyclic aromatic hydrocarbons such as, for example, phenyl, 1-naphthyl, 2-naphthyl anthracenyl, phenanthrenyl, and the like. In some embodiments, aryl groups have from 6 to about 20 carbon atoms.

As used herein, “arylalkyl” or “aralkyl” refers to a group of formula -alkyl-aryl. Preferably, the alkyl portion of the arylalkyl group is a lower alkyl group, i.e., a C₁₋₆ alkyl group, more preferably a C₁₋₃ alkyl group. Examples of aralkyl groups include benzyl and naphthylmethyl groups.

As used herein, “halo” or “halogen” includes fluoro, chloro, bromo, and iodo.

At various places in the present specification substituents of compounds of the invention are disclosed in groups or in ranges. It is specifically intended that the invention include each and every individual subcombination of the members of such groups and ranges. For example, the term “C₁₋₄ alkyl” is specifically intended to individually disclose methyl, ethyl, propyl, isopropyl, n-butyl, sec-butyl, isobutyl, etc.

The compounds of the present invention may contain an asymmetric carbon atom and some of the compounds of this invention may contain one or more asymmetric centers and may thus give rise to optical isomers and diastereomers. While shown without respect to stereochemistry in the compounds of the present invention, the compounds of the present invention include such optical isomers and diastereomers; as well as the racemic and resolved, enantiomerically pure R and S stereoisomers; as well as other mixtures of the R and S stereoisomers and pharmaceutically acceptable salts thereof.

Where a stereoisomer is preferred, it may in some embodiments be provided substantially free of the corresponding enantiomer. Thus, an enantiomer substantially free of the corresponding enantiomer refers to a compound that is isolated or separated via separation techniques or prepared free of the corresponding enantiomer. “Substantially free”, as used herein, means that the compound is made up of a significantly greater proportion of one steriosomer, preferably less than about 50%, more preferably less than about 75%, more preferably less than about 90%, and even more preferably less than about 95%.

As used herein, acceptable salts of the compounds having the structure of Formula I with an acidic moiety can be formed from organic and inorganic bases. Suitable salts with bases are, for example, metal salts, such as alkali metal or alkaline earth metal salts, for example sodium, potassium, or magnesium salts; or salts with ammonia or an organic amine, such as morpholine, thiomorpholine, piperidine, pyrrolidine, a mono-, di- or tri-lower alkylamine, for example ethyl-tert-butyl-, diethyl-, diisopropyl-, triethyl-, tributyl- or dimethylpropylamine, or a mono-, di-, or trihydroxy lower alkylamine, for example mono-, di- or triethanolamine. Internal salts may furthermore be formed. Similarly, when a compound of the present invention contains a basic moiety, salts can be formed from organic and inorganic acids. For example salts can be formed from acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known pharmaceutically acceptable acids.

The present invention also provides compounds that are useful as intermediates in the preparation of compounds of Formula I. In some embodiments, the compounds have the Formula IIa: 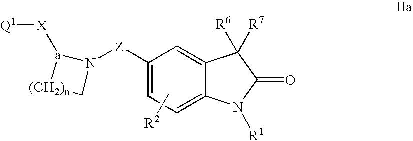
or a salt thereof; wherein:

R¹ is H, C₁-C₆ alkyl or C₇-C₂₄ aralkyl;

R² is H, halogen or C₁-C₆ alkyl;

n is 1, 2 or 3;

X is —(CH₂)_{q}—;

q is 0, 1, 2 or 3;

R⁶ and R⁷ are each independently OH or —O—(C₁-C₆ alkyl);
or R⁶ and R⁷ taken together can form ═O or —O—(CH₂)ᵣ—O—, where r is 2 or 3;

Z is CO or SO₂;

Q is N(R¹⁰)(R¹¹);

R¹⁰ and R¹¹ are each independently H or a protecting group;
or R¹⁰ and R¹¹ taken together with the nitrogen atom to which they are attached can form an N-phthalimidyl group; and

a is a carbon atom.

In some embodiments, the carbon atom designated “a” has the S configuration. In some embodiments, R¹ is hydrogen. In some embodiments, R⁶ and R⁷ taken together form —O—(CH₂)ᵣ O—, where r is 2 or 3. In some embodiments, n is 2. In some embodiments, X is —CH₂—. In some embodiments, R⁶ and R⁷ are each H.

In some preferred embodiments, R¹ and R² are each H; n is 2; X is —CH₂—; R⁶ and R⁷ together form ═O; and R¹⁰ and R¹¹ taken together with the nitrogen atom to which they are attached form an N-phthalimidyl group.

In some preferred embodiments, R¹ and R² are each H; n is 2; X is —CH₂—; R⁶ and R⁷ together form —O—(CH₂)₃—O—; and R¹⁰ and R¹¹ taken together with the nitrogen atom to which they are attached form an N-phthalimidyl group.

In some preferred embodiments, R¹ is methyl; R² is H; n is 2; X is —CH₂—; R⁶ and R⁷ together form —O—(CH₂)₃—O—; and R¹⁰ and R¹¹ taken together with the nitrogen atom to which they are attached form an N-phthalimidyl group.

In some preferred embodiments, R¹ is methyl; R² is H; n is 2; X is —CH₂—; R⁶ and R⁷ together form —O—(CH₂)₃—O—; and Q is NH₂.

In one aspect, the present invention provides fluorescent polarization assays for the detection and evaluation of potential caspase inhibitors. The assays of the present invention utilize the measurement of fluorescence polarization of a fluorescence-emitting compound that binds to the active site of a caspase.

In some embodiments, the present invention provides fluorescence polarization assays for determining whether a test compound binds to a caspase, comprising:

(a) combining the test compound, a fluorescent probe, and a caspase in a solution;

(b) incubating the solution until equilibrium has been reached; and

(c) determining the difference in the amount of probe bound o the caspase in the presence and absence of the test compound;

wherein the probe is a compound of the invention of Formula I, as described herein.

In some embodiments, the concentration of caspase in the solution is selected to provide a preselected value of fraction of probe bound to the caspase. In some such embodiments, the concentration of caspase in the solution is selected by incubating the probe with varying concentrations of caspase; measuring fluorescence polarization values for the varying concentrations of caspase; converting the fluorescence polarization values (i.e., the fluorescence polarization intensities) to values of fraction bound; and selecting an enzyme concentration providing a desired value of fraction bound. In some embodiments, the fluorescence polarization values are converted to anisotropy values, which are then converted to values of fraction of probe bound to the caspase. In some embodiments, anisotropy values are determined directly from the measured fluorescence polarization values, and then converted to values of fraction bound. In some embodiments, the measured fluorescence polarization values are first converted to MilliP values, which are then converted to anisotropy values, which are in turn converted to values of fraction bound.

In some embodiments, step (c) includes determining the fluorescence polarization value of the fluorescent probe in the solution; determining the fraction of probe bound to the caspase from the fluorescence polarization value; and comparing the fraction of probe bound to the caspase to the fraction of probe bound to the caspase in the absence of the test compound.

In some embodiments, step (a) includes contemporaneously combining together the caspase, the probe, and the test compound. In some further embodiments, step (a) includes combining the caspase and the test compound together to form a mixture; waiting for a period of time; and adding the probe to the mixture. In some further embodiments, step (a) includes combining the caspase and the probe together to form a mixture; waiting for a period of time; and adding the test compound to the mixture.

In some embodiments, the methods further include performing the steps of the methods a plurality of times, each with a different concentration of test compound, and determining a Ki value for the test compound.

In some embodiments, the invention provides fluorescence polarization assays for screening a plurality of test compound for binding to a caspase. In some embodiments, the assays include:

(a) providing a plurality of test solutions, each containing a test compound, a fluorescent probe, and a caspase;

(b) incubating the solutions until equilibrium has been reached; and

(c) determining the differences in the amounts of probe bound to the caspase in the presence and absence of the test compounds;

wherein the probe is a compound of the invention of Formula I, as described herein.

In some embodiments, the concentration of caspase in the test solutions is selected to provide a preselected value of fraction of probe bound to the caspase. In some such embodiments, the selection of the concentration of caspase in the test solutions includes:

i) incubating the probe with varying concentrations of caspase;

ii) measuring fluorescence polarization values for the varying concentrations of caspase;

iii) converting the measured fluorescence polarization values to values of fraction bound; and

iv) selecting an caspase concentration providing a desired value of fraction of probe bound to the caspase.

In some embodiments, step (iii) includes converting the fluorescence polarization values to anisotropy values.

In some embodiments, for each test solution, step (c) includes:

(c₁) determining the fluorescence polarization value of the fluorescent probe in the test solution;

(c₂) determining the fraction of probe bound to the caspase from the fluorescence, polarization values; and

(c₃) comparing the fraction of probe bound to the caspase to the fraction of probe bound to the caspase in the absence of the test compound.

In some such embodiments, for each test solution, step (c₂) includes converting the fluorescence polarization values to anisotropy values.

In some embodiments, for each test solution, step (a) includes contemporaneously combining together the caspase, the probe, and the test compound. In some embodiments, for each test solution, step (a) includes combining the caspase and the test compound together to form a mixture; waiting for a period of time; and adding the probe to the mixture. In some embodiments, for each test solution, step (a) includes combining the caspase and the probe together to form a mixture; waiting for a period of time; and adding the test compound to the mixture.

In some embodiments, the methods further include performing the steps (a)-(c) a plurality of times, each with a different concentration of test compound, and determining a Ki value for each test compound.

Preparation of Compounds of the Invention

The compounds of present invention can be conveniently prepared in accordance with the procedures outlined in the schemes below, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures. Those skilled in the art of organic synthesis will recognize that the nature and order of the synthetic steps presented may be varied for the purpose of optimizing the formation of the compounds of the invention.

The processes described herein can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatograpy (HPLC) or thin layer chromatography.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

General Schemes for Preparation of Compounds of the Invention

The compounds of the present invention can be prepared according to the synthetic routes shown in Schemes 1-4. As shown in Schemes 1-4, compounds having the structure of Formula I wherein R¹, R², R³, R⁴, R⁵, n, m, q, X and Z are as defined above can be prepared from an alcohol Compound III wherein Y is (CH₂)_{q}, P¹ is a protecting group for amine functionality such as t-butoxcarbonyl (Boc) and q is as defined above. The starting alcohol of Compound III may be either commercially available such as (S)-(−)-1-(t-butoxycarbonyl)-2-pyrrolidine methanol, or synthesized by selectively protecting the amine functionality of an alcohol compound having a cylcoamine moiety with an appropriate reagent such as (Boc)₂O.

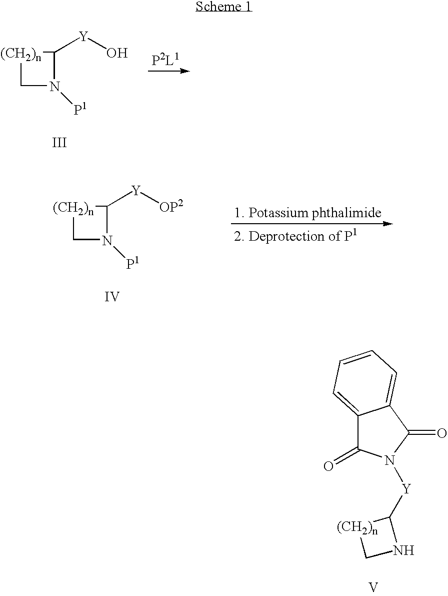

As shown in Scheme 1, the conversion of Compound III having the OH group, to Compound IV having the OP², a good leaving group such as —O-Tosyl, is achieved by reacting Compound III with a suitable reagent such as Tosyl chloride, in the presence of base such as pyridine, in solvent such as methylene chloride at temperatures ranging from 0° C. to room temperature. The conversion of Compound IV to Compound V is achieved by (1) treating Compound IV with potassium phthalimide to displace the OP² group with phthalimide group, in solvent such as DMF at an elevated temperature such as 60° C.; followed by (2) deprotection of the amine group on the cylcoamine moiety with a suitable reagent such as hydrochloric acid in solvent such as ethanol where the protecting group is Boc.

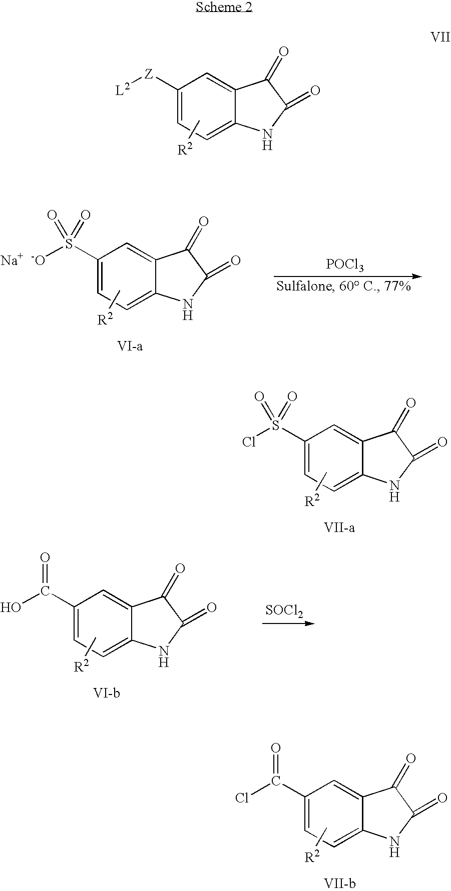

Compound VII where Z is defined as above and L² is a leaving group such as chloride can be synthesized according to Scheme 2. Where Z is sulfonyl, a sulfonic acid salt compound VI-a is reacted with POCl₃ in a solvent such sulfalone at an elevated temperature such as 60° C. to afford a sulfonic acid chloride Compound VII-a. Where Z is carbonyl group, an acid chloride Compound VII-b can be conveniently obtained by reacting a carboxylic acid compound VI-b with SOCl₂.

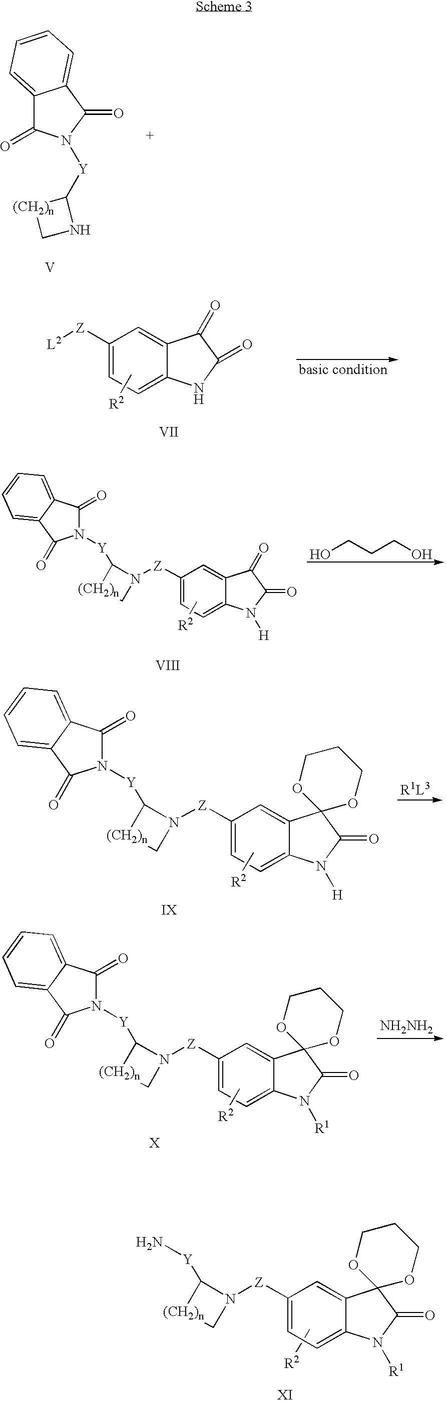

As shown in Scheme 3, coupling of cycloamine Compound V with (sulfonic) acid chloride Compound VII in the presence of base such as diisopropylethylamine in a solvent such as methylene chloride at low temperatures (0° C.) produces Compound VII. The ketone in Compound VII can be protected to prevent unwanted side reactions by using a suitable protecting group such as an acyclic (dimethyl or diisopropyl) or cyclic (dioxolane, dioxane) ketal. In this scheme, protection of the ketone in Compound VII is accomplished using a suitable protecting agent such as 1,3-propanediol in the presence of an acid such as H₂SO₄ or p-toluenesulfonic acid under refluxing conditions in benzene or toluene while azeotropically removing H₂O with a Dean-Stark trap to give a ketal of Compound IX. The species of the ketone protecting group employed is not critical so long as the derivatized ketone is stable to the conditions of subsequent reaction(s) and can be removed at the appropriated point without disrupting the remainder of the molecule. Further examples of these groups are found in T. W. Greene and P. G. M. Wuts, “Protective Groups in Organic Synthesis,” 2^{nd} ed. John Wiley and Sons, New York, N.Y., Chapter 4, incorporated herein by reference. Alkylating the protected Compound IX to produce Compound X utilizing the desired alkylating reagent of compound R¹L³, where R¹ is defined above and L³ is a leaving group such as iodide, is accomplished by using a suitable base such as NaH or K₂CO₃ in a nonprotic solvent such as THF at temperatures ranging from 0° C. to reflux. The alkylating reagent is either commercially available or is prepared using standard synthetic methodology as described by Cliffe, I. A., Syn. Comm. 20(12) 1757, 1990. The phthalimide Compound X is reacted with hydrazine by refluxing in solvent such as methanol to afford the free amine Compound XI.

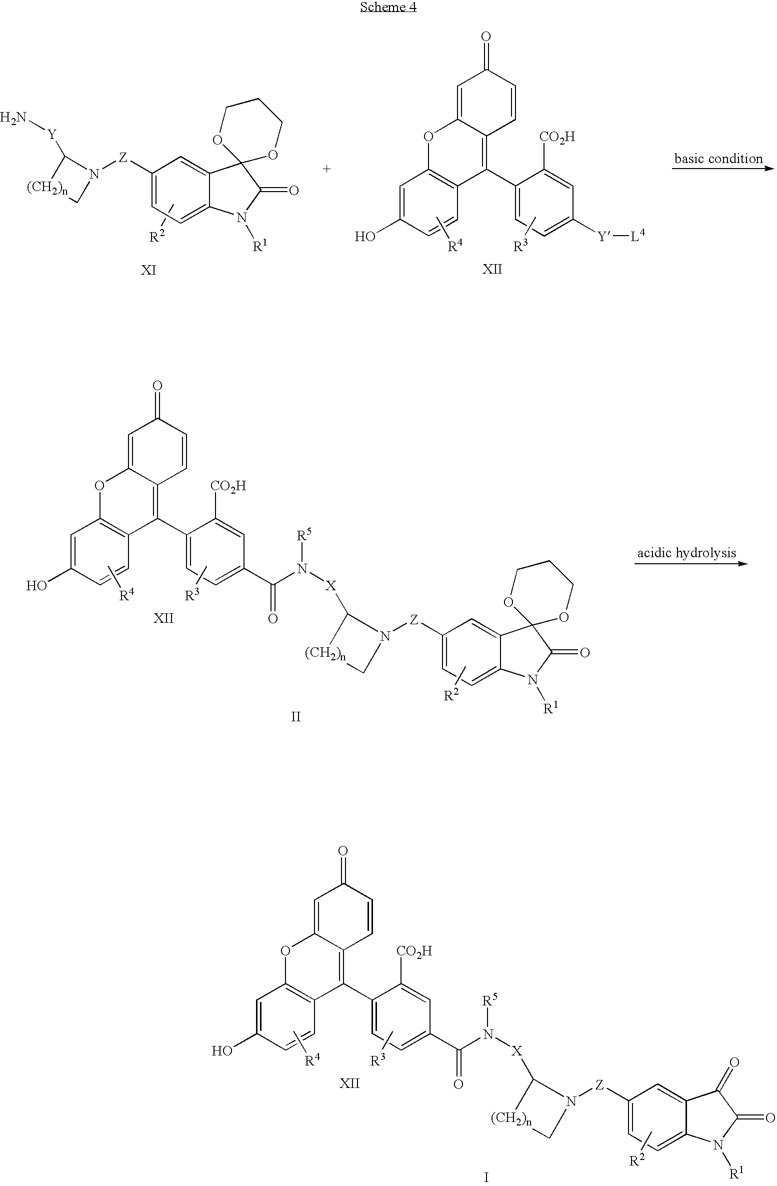

As shown in Scheme 4, Compound XII has the structure of Formula XII wherein L⁴ is a leaving group such as succinimidyloxy; Y′ is C(O), C(O)—NR⁵—(CH₂)ₘ—C(O), or C(O)—NR⁵—-(CH₂)ₘ−SO₂; and R³, R⁴, R⁵ and m are defined above. Compound XII such as 5-carboxyfluorescein, succinimidyl ester (5-FAM) or 6-(fluorescein-5-carboxamido)hexanoic acid, succinimidyl ester (5-SFX) can be commercially available from Molecular Probes, a division of Invitrogen Corporation. Coupling of amine Compound XI with Compound XII in solvent such as DMF produces Compound II. The ketal in Compound II can be removed by acidic hydrolysis utilizing an acid such as H₂SO₄ or CH₃SO₄ either neat or utilizing a suitable organic solvent such CH₂Cl₂ at temperatures ranging from 0° C. to 80° C. to produce compounds having the structure of Formula I.

Preparation of Fluorescence Probes

### EXAMPLE 1

### 2-(6-Hydroxy-3-oxo-3H-xanthen-9-yl)-5-{[({(2S)-1-[(1-methyl-2,3-dioxo-2,3-dihydro-1H-indol-5-yl)sulfonyl]pyrrolidin-2-yl}methyl)amino]carbonyl}benzoic acid

Step 1: (S)—N-Boc-2-(4-Toluenesulfonyloxymethyl)pyrrolidine

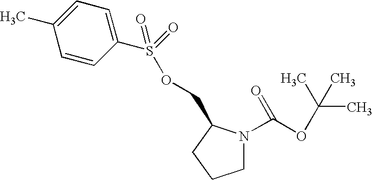

At 0° C., to a stirred solution containing (S)-(−)-1-(t-butoxycarbonyl)-2-pyrrolidine methanol (5.23 g, 26.0 mmol) and pyridine (14.7 mL, 0.182 mol) in CH₂Cl₂ (26 mL) was added dropwise a solution of p-toluenesulfonyl chloride in CH₂Cl₂ (31 mL). After the addition was complete, the ice bath was removed and the reaction was stirred for 2 days. The reaction was quenched with H₂O (150 mL) and extracted with ether. The combined ethereal extracts were washed successively with sat. aq. CuSO₄ (3×), with H₂O (1×), with brine (3×), dried (Na₂SO₄) and concentrated to give 8.62 g, (93%) of the title compound as an oil. ¹H NMR (DMSO-d₆) yielded spectra consistent with the assigned structure.
Step 2: tert-Butyl (2S)-2-[(1,3-dioxo-1,3-dihydro-2H-isoindol-2-yl)methyl]pyrrolidine-1-carboxylate

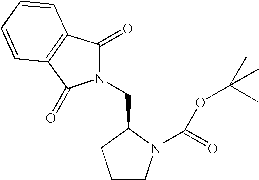

A stirred mixture containing (S)—N-Boc-2-(4-toluenesulfonyloxymethyl)pyrrolidine (9.02 g, 25.4 mmol) and potassium phthalimide (5.17 g, 27.9 mmol) in N,N-DMF (127 mL) was heated at 60° C. for 4 hours. The reaction was cooled to room temperature and stirred for 3 days. Analytical HPLC indicated that the reaction was incomplete. The reaction was heated at 70° C. for an additional 18 hours. The reaction was cooled to room temperature, quenched with H₂O (1 L) and extracted with ether. The combined ethereal extracts were washed successively with 1 N NaOH (3×), with brine (3×), dried (K₂CO₃) and concentrated to give 3.10 g, (37%) of the title compound as an oil. ¹H NMR (DMSO-d₆) yielded spectra consistent with the assigned structure.
Step 3: 2-[(2S)-Pyrrolidin-2-ylmethyl]-1H-isoindole-1,3(2H)-dione hydrochloride

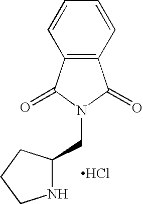

At room temperature, tert-butyl (2S)-2-[(1,3-dioxo-1,3-dihydro-2H-isoindol-2-yl)methyl]pyrrolidine-1-carboxylate was stirred in 9M ethanolic HCl for 2 hours. The reaction was diluted with ether (500 mL). The product precipitated and was collected and dried to give 2.10 g, (84%) of the title compound. ¹H NMR (DMSO-d₆) yielded spectra consistent with the assigned structure.
Step 4: 5-({(2S)-2-[(1,3-Dioxo-1,3-dihydro-2H-isoindol-2-yl)methyl]pyrrolidin-1-yl}sulfonyl)-1H-indole-2,3-dione

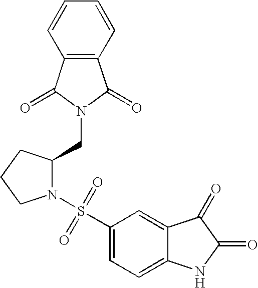

At 0° C., to a stirred suspension containing 5-chlorosulfonylisatin (1.76 g, 7.15 mmol) (J. Med. Chem, 44, 2014, 2001) and 2-[(2S)-pyrrolidin-2-ylmethyl]-1H-isoindole-1,3(2H)-dione hydrochloride (2.10 g, 7.83 mmol) in CH₂Cl₂ (36 mL) was added dropwise diisopropylethylamine (3.74 mL, 21.4 mmol). After the addition was complete, the reaction was stirred for 16 hours. The reaction was quenched with H₂O (100 mL) and extracted with CH₂Cl₂. The combined organic extracts were dried (MgSO₄) and concentrated. The crude product was purified on Biotage KP-Sil eluting with 15% acetone/CH₂Cl₂ to give 2.81 g, (90%) of the title compound. ¹H NMR (DMSO-d₆) yielded spectra consistent with the assigned structure.
Step 5: 2-({(2S)-1-[(2′-Oxo-1′,2′-dihydrospiro[1,3-dioxane-2,3′-indol]-5′-yl)sulfonyl]pyrrolidin-2-yl}methyl)-1H-isoindole-1,3(2H)-dione

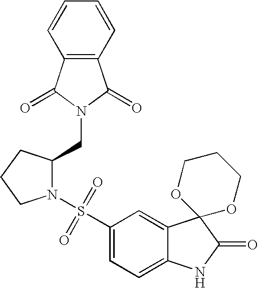

A stirred mixture containing 5-({(2S)-2-[(1,3-Dioxo-1,3-dihydro-2H-isoindol-2-yl)methyl]pyrrolidin-1-yl}sulfonyl)-1H-indole-2,3-dione (2.73 g, 6.22 mmol), 1,3-propanediol (1.80 mL, 24.9 mmol) and p-toluene sulfonic acid monohydrate (0.236 g, 1.24 mmol) in benzene (130 mL) was refluxed with a Dean-Stark trap for 20 hours. The reaction was cooled to room temperature, diluted with EtOAc (100 mL) and CH₂Cl₂ (40 mL), washed successively with H₂O (2×), with brine (2×), dried (Na₂SO₄) and concentrated. The crude product was purified on Biotage KP-Sil eluting with 10% acetone/CH₂Cl₂ to give 1.59 g, (51%) of the title compound. ¹H NMR (DMSO-d₆) yielded spectra consistent with the assigned structure.
Step 6: 2-({(2S)-1-[(1′-Methyl-2′-oxo-1′,2′-dihydrospiro[1,3-dioxane-2,3′-indol]-5′-yl)sulfonyl]pyrrolidin-2-yl}methyl)-1H-isoindole-1,3(2H)-dione

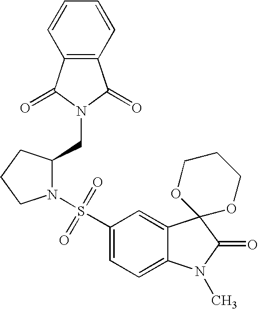

At room temperature, to a stirred suspension containing 2-({(2S)-1-[(2′-oxo-1′,2′-dihydrospiro[1,3-dioxane-2,3′-indol]-5′-yl)sulfonyl]pyrrolidin-2-yl}methyl)-1H-isoindole-1,3(2H)-dione (1.58 g, 3.17 mmol) and K₂CO₃ (1.09 g, 7.93 mmol) in N,N-DMF (32 mL) was added iodomethane (0.901 g, 6.35 mmol). After 18 hours, the reaction was diluted with H₂O (250 mL) and the resulting precipitate collected by filtration and dried to give 1.37 g, (84%) of the title compound. ¹H NMR (DMSO-d₆) yielded spectra consistent with the assigned structure.
Step 7: 5′-{[(2S)-2-(Aminomethyl)pyrrolidin-1-yl]sulfonyl}-1′-methylspiro[1,3-dioxane-2,3′-indol]-2′(1′H)-one

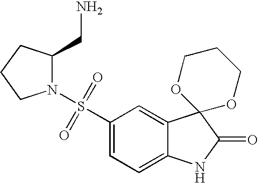

A stirred suspension containing 2-({(2S)-1-[(1′-methyl-2′-oxo-1′,2′-dihydrospiro[1,3-dioxane-2,3′-indol]-5′-yl)sulfonyl]pyrrolidin-2-yl}methyl)-1H-isoindole-1,3(2H)-dione (1.37 g, 2.68 mmol) and hydrazine (0.629 mL) in MeOH (100 mL) was refluxed for 18 hours. The reaction was cooled to room temperature, quenched with H₂O (100 mL) and extracted with EtOAc. The combined organic extracts were washed with brine (3×), dried (Na₂SO₄) and concentrated. The crude product was purified on Biotage KP-Sil eluting with CH₂Cl₂:MeOH:NH₄OH (92.5:5:2.5) to give 0.871 g, (85%) of the title compound as a white solid. mp 75-80° C.; ¹H NMR (DMSO-d₆) yielded spectra consistent with the assigned structure, although one proton was obscured by water resonance at 3.3 ppm. MS (ES) m/z 382.2
Step 8: 2-(6-Hydroxy-3-oxo-3H-xanthen-9-yl)-5-{[({(2S)-1-[(1′-methyl-2′-oxo-1′,2′-dihydrospiro[1,3-dioxane-2,3′-indol]-5′-yl)sulfonyl]pyrrolidin-2-yl}methyl)amino]carbonyl}benzoic acid

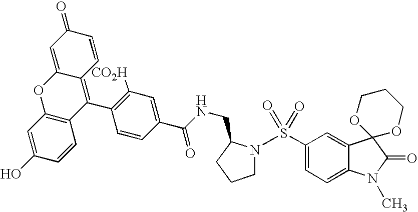

At room temperature, to a stirred solution of 5-carboxyfluorescein succinimidyl ester (20 mg, 0.0423 mmol) (Molecular Probes, Inc.) in N,N-DMF (1.6 mL) was added dropwise a solution of 5′-{[(2S)-2-(aminomethyl)pyrrolidin-1-yl]sulfonyl}-1′-methylspiro[1,3-dioxane-2,3′-indol]-2′(1′H)-one (19.3 mg, 0.0507 mmol) in N,N-DMF (0.507 mL). After 18 hours, the reaction was quenched with H₂O (20 mL) and extracted with EtOAc. The combined organic extracts were washed with brine (3×), dried (Na₂SO₄) and concentrated to give 35 mg of 2-(6-hydroxy-3-oxo-3H-xanthen-9-yl)-5-{[({(2S)-1-[(1′-methyl-2′-oxo-1′,2′-dihydrospiro[1,3-dioxane-2,3′-indol]-5′-yl)sulfonyl]pyrrolidin-2-yl}methyl)amino]carbonyl}benzoic acid. This material was used without any additional purification. MS (ES) m/z 740.5
Step 9: 2-(6-Hydroxy-3-oxo-3H-xanthen-9-yl)-5-{[({(2S)-1-[(1-methyl-2,3-dioxo-2,3-dihydro-1H-indol-5-yl)sulfonyl]pyrrolidin-2-yl}methyl)amino]carbonyl}benzoic acid

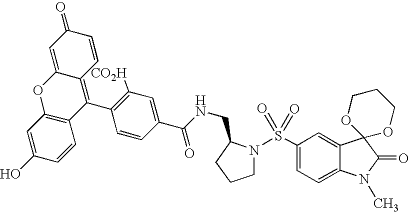

At room temperature, 2-(6-hydroxy-3-oxo-3H-xanthen-9-yl)-5-{[({(2S)-1-[(1′-methyl-2′-oxo-1′,2′-dihydrospiro[1,3-dioxane-2,3′-indol]-5′-yl)sulfonyl]pyrrolidin-2-yl}methyl)amino]carbonyl}benzoic acid (35 mg) was stirred in methanesulfonic acid (2 mL) for 1 hour. Analytical LC/MS indicated that the ketal was consumed. The reaction was quenched with crushed ice and the resulting precipitate collected by filtration, washed with H₂O and dried to give 22 mg (76%) of the title compound. ¹H NMR (DMSO-d₆) yielded spectra consistent with the assigned structure, although several peaks were present under H₂O peak. MS (ES) m/z 682.1; MS (ES) m/z 714.2;

### EXAMPLE 2

### 2-(6-Hydroxy-3-oxo-3H-xanthen-9-yl)-5-[({6-[({(2S)-1-[(1-methyl-2,3-dioxo-2,3-dihydro-1H-indol-5-yl)sulfonyl]pyrrolidin-2-yl}methyl)amino]-6-oxohexyl}amino)carbonyl]benzoic acid

Step 1: 2-(6-Hydroxy-3-oxo-3H-xanthen-9-yl)-5-[({6-[({(2S)-1-[(1′-methyl-2′-oxo-1′,2′-dihydrospiro[1,3-dioxane-2,3′-indol]-5′-yl)sulfonyl]pyrrolidin-2-yl}methyl)amino]-6-oxohexyl}amino)carbonyl]benzoic acid

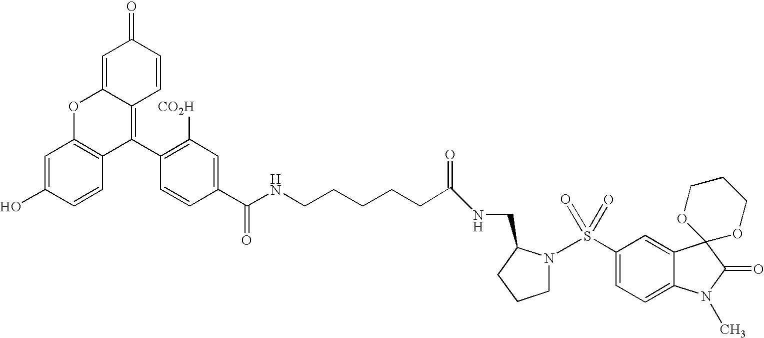

At room temperature, to a stirred solution of 6-(fluorescein-5-carboxamido)hexanoic acid succinimidyl ester (20 mg, 0.0341 mmol) (Molecular Probes, Inc.) in N,N-DMF (0.800 mL) was added dropwise 0.1 M 5′-{[(2S)-2-(aminomethyl)pyrrolidin-1-yl]sulfonyl}-1′-methylspiro[1,3-dioxane-2,3′-indol]-2′(1′H)-one in N,N-DMF (0.409 mL, 0.0409 mmol). After 18 hours, the reaction was quenched with H₂O (20 mL) and extracted with EtOAc. The combined organic extracts were washed with brine (3×), dried (Na₂SO₄) and concentrated to give 28 mg, (96%) of the title compound. MS (ES) m/z 583.6
Step 2: 2-(6-Hydroxy-3-oxo-3H-xanthen-9-yl)-5-[({6-[({(2S)-1-[(1-methyl-2,3-dioxo-2,3-dihydro-1H-indol-5-yl)sulfonyl]pyrrolidin-2-yl}methyl)amino]-6-oxohexyl}amino)carbonyl]benzoic acid

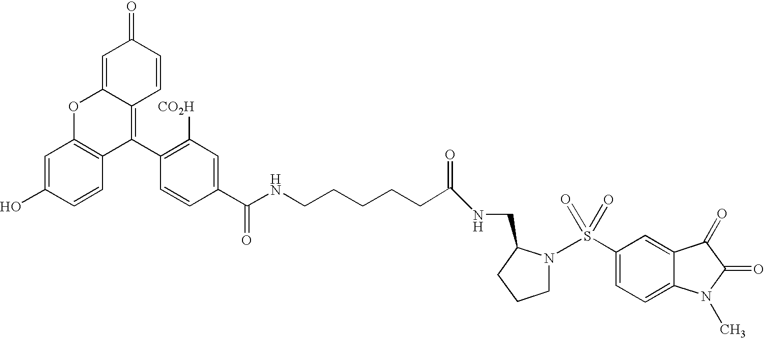

At room temperature, 2-(6-hydroxy-3-oxo-3H-xanthen-9-yl)-5-[({6-[({(2S)-1-[(1′-methyl-2′-oxo-11′,2′-dihydrospiro[1,3-dioxane-2,3′-indol]-5′-yl)sulfonyl]pyrrolidin-2-yl}methyl)amino]-6-oxohexyl}amino)carbonyl]benzoic acid (28 mg, 0.0328 mmol) was stirred in methanesulfonic acid (2 mL) for 1 hour. Analytical LC/MS indicated that the ketal was consumed. The reaction was quenched with crushed ice and the resulting precipitate collected by filtration, washed with H₂O and dried to give 19.2 mg (74%) of the title compound. ¹H NMR (DMSO-d₆) yielded spectra consistent with the assigned structure. MS (ES) m/z 795.3

### EXAMPLE 3

Preparation of Active Caspase 3

Caspase 3 was expressed intracellularly in E. coli with a c-terminal His tag. Fermentation was performed at 25° C. in a B. Braun Biotech Biostat C 10 liter bioreactor vessel. The culture was collected in 1 L bottles and centrifuged in Komspin KA-7.1000 rotors at approximately 8000 RCF (Relative Centrifugal Force). The cell pellets were re-suspended in 20 mM Tris pH 8.0, 500 mM NaCl, and 5 mM imidazole. The cell suspension was disrupted by passing 5 times through a microfluidizer Model 110Y (Microfluidics Corp, Newton, Mass.). After centrifugation (13 kg, 30 min at 4° C.), the supernatant was applied to a column of Nickle-NTA agarose. The Caspase 3 was eluted with a gradient of 5 mM to 150 mM imidazole in the above buffer. Fractions containing Caspase 3 were pooled and concentrated with a Millipore Ultrafree filtration device. The concentrated Caspase 3 solution was loaded unto a TSK gel G3000sw column (Tosoh Bioseph LLC), equilibrated with a buffer of 20 mM PIPES pH 7.2, 100 mM NaCl, 1 mM EDTA & 5 mM Cysteine. Fractions containing Caspase 3 were pooled and concentrated. Sometimes CHAPS was added to 0.1% and sucrose to 10% into the protein sample. The Caspase 3 obtained with this method shows two subunits of 17 and 13 kD on reduced SDS-PAGE and aliquots stored at −80° C.

### EXAMPLE 4

Caspase 3 Inhibition Assay

This standard pharmacological test procedure to assess the inhibition of recombinant caspase 3 activity of selected compounds was adapted from Thornbery, N., J. Biol. Chem. 1997 272(29) 17907-17911 and Stennicke, H. R. et al., J. Biol. Chem. 1997 272(41) 25719-25723. The procedure used and results obtained are briefly described below.

Caspase 3 was assayed at 23° C. (room temp) in 96-well plates using the internally quenched tetrapeptide substrate N-acetyl-aspartyl-glutamyl-valyl-aspartate-7-amino-4-trifluoromethyl coumarin (Ac-DEVD-AFC purchased from Biomol). The assays are conducted at pH 7.2 in a buffered system containing 20 mM PIPES, 100 mM NaCl, 1 mM EDTA, 0.1% CHAPS, 10% sucrose and 5 mM L-cysteine. The final concentration of the substrate is 25 μM. Enzymic cleavage between the aspartate and the AFC fluorophore liberates 7-amino-4-trifluoromethyl coumarin which is detected using an excitation wavelength of 400 nm and an emission wavelength of 505 nm in a SpectraMax GeminiXS plate reader operated at room temperature. A steady state rate of substrate cleavage is obtained for analysis.

For IC₅₀ determinations, typically 11 concentrations ranging from 20 μM to 20 nM were prepared by serial dilution with assay buffer containing no cysteine with 80 ul of 31.25 μM substrate added to the assay well. Once substrate and inhibitor were added to the assay plate, the reaction was initiated by addition of 10 μl of 2.5 nM enzyme, prepared in assay buffer containing 50 mM Cysteine, to the assay mixture (final concentration 0.25 nM). After the reaction was initiated with the addition of enzyme, AFC production was monitored continuously for 90 minutes by exciting at 400 nm and measuring the emission at 505 nm every 42 seconds. The progress curves generated were fitted by computer to equation 1 to generate an IC50 value.
y=Bmax*(1−(xⁿ/(Kⁿ+xⁿ))), where Bmax is rate in the absence of inhibitor. Equation 1

If only one concentration of inhibitor was tested or the IC50 was greater than the highest concentration of compound used in the assay, the percent inhibition at the highest compound concentration is reported.

### EXAMPLE 5

Fluorescence Polarization Assay (Caspase Inhibition Assay)

A. Determination of Probe IC50

The substrate Ac-DEVD-AFC (25 μM) was added to varying inhibitor (probe) concentrations ranging from 20 μM to 2 nM. A caspase (250 μM) was added last to initiate the enzymatic reaction. The cleavage of the substrate by the enzyme (caspase) was monitored continuously for 90 minutes by exciting the substrate at 400 nm and monitoring the fluorescence at emission at 505 nm. The rate was fitted to the sigmoidal inhibition equation below to obtain an IC₅₀:
y=Bmax*(1−(xˆn/(Kiˆn+xˆn))) Eq. 3
wherein:
y is the observed value of the cleavage rate,
Bmax is the cleavage rate in the absence of inhibitor,
x is the substrate concentration,
n is the number of the active binding sites of the enzyme (caspase), and
Ki is the inhibition constant of the inhibitor (probe).

Using the method above, the IC50 for 2-(6-Hydroxy-3-oxo-3H-xanthen-9-yl)-5-[({6-[({(2S)-1-[(1-methyl-2,3-dioxo-2,3-dihydro-1H-indol-5-yl)sulfonyl]pyrrolidin-2-yl}methyl)amino]-6-oxohexyl}amino)carbonyl]benzoic acid was determined to be 15 nM, and the IC50 for 2-(6-Hydroxy-3-oxo-3H-xanthen-9-yl)-5-{[({(2S)-1-[(1-methyl-2,3-dioxo-2,3-dihydro-1H-indol-5-yl)sulfonyl]pyrrolidin-2-yl}methyl)amino]carbonyl}benzoic acid was determined to be 10 nM.

B. Determination of Probe Anisotropy

A probe compound of the invention (5 nM) was incubated with various concentrations (488 μM to 1000 nM) of a caspase in a buffer solution. Fluorescent polarization (MilliP) and intensity readings were recorded over a period of 150 minutes. 5 nM ligand alone yielded a MilliP value of 20. Fully bound, the MilliP value was ˜300. The MilliP value was converted to Anisotropy (the “A” value) according to the equation A=2*P/(3−P) where P=1000 milliP. The anisotropy was converted to a fraction bound value (Fb) value according to equation 4, wherein Q is determined according to equation 5:

$$\begin{matrix}Fb = \frac{{A}_{obs} - {A}_{o}}{( {A}_{max} - {A}_{obs} ) Q + {A}_{obs} - {A}_{o}} & \text{Eq. 4} \\ Q = \frac{Saturating\_Enzyme \_Intensity}{Ligand\_Alone \_Intensity} & \text{Eq. 5}\end{matrix}$$

A caspase concentration was identified that provided approximately 70% fraction bound for the displacement assay.

C. Competitive Displacement Assay

The present invention is also directed to fluorescence polarization assays to provide a reliable quantitative means for measuring the amount of probe-caspase complex produced in a homogeneous competitive binding assay. Typically, in such a competitive binding assay a test compound (a potential caspase inhibitor to be determined by the technique) competes with a fluorescently labeled reagent, or “fluorescent probe”, for an active binding site on a caspase. The concentration of the fluorescent probe in the sample determines the amount of the test compound which binds to the caspase: the amount of the test compound that will bind is inversely proportional to the concentration of the fluorescent probe in the sample, because the fluorescent probe and the test compound each bind to the caspase in proportion to their respective concentrations.

Fluorescence polarization techniques are based on the principle that a fluorescent labeled compound, when excited by plane polarized light, will emit fluorescence having a degree of polarization inversely related to its rate of rotation. Accordingly, when a probe-caspase complex having a fluorescent label, for example, is excited with plane polarized light, the emitted light remains highly polarized because the fluorophore is constrained from rotating between the time that light is absorbed and emitted. In contrast, when a “free” fluorescent probe compound (i.e., unbound to a caspase) is excited by plane polarized light, its rotation is much faster than that of the corresponding probe-caspase conjugate and the molecules are more randomly oriented. As a result, the light emitted from the unbound probe molecules is depolarized.

The present inventors have discovered that the novel fluorescent probes of the present invention can be used in a fluorescence polarization assay to detect and evaluate caspase inhibitors. The fluorescent probes of the present invention specifically bind to an active site of a caspase. Upon complexing with the caspase, the probe-caspase complex thus formed assumes the rotation of the probe molecule which is slower than that of the relatively small fluorescent probe molecule, thereby increasing the polarization observed. When a potential caspase inhibitor test compound competes with the fluorescent probe for the active site of the caspase, less probe-caspase complex is formed, i.e., there is more probe in an uncomplexed, free form. Therefore, the observed polarization of fluorescence of the resulting mixture of free probe and probe-caspase complex assumes a value intermediate between that of the free probe and that of the probe-caspase complex. Thus, there is a reduction of the fluorescence polarization value in the presence of a competitive caspase inhibitor as compared to when no such inhibitor is present. Inhibitor dissociation constant can then be easily determined in order to evaluate the relative strength of the competitive caspase inhibitor.

The fluorescence polarization data can be converted to the fraction bound value according to Eqs 1-5 and the resulting fraction bound data can be fitted into the following equation to obtain the dissociation constant K_{d} for the test compound (a competitive inhibitor):
Y=(1+Ki/Kd*((1−x)/x))(Enz−Kd*x/(1−x)−L*x) Eq. 6
wherein:
Y is fraction bound value;
L is ligand (probe) concentration;
Enz is enzyme (caspase) concentration;
Ki is the inhibition constant of the probe; and
x is inhibitor concentration.

Unless otherwise specified herein, the conditions that can be employed in running the fluorescence polarization assays of the present invention (e.g., pressure, temperature, pH, solvents, buffer, time) may be readily determined by one having ordinary skill in the art. One skilled in the art will understand that the optimum assay conditions may vary depending on the particular reagents used (i.e., the fluorescent probe, the caspase and the test compound) and such optimum conditions can also be readily determined by one skilled in the art based on the general knowledge in the field of fluorescence polarization assays.

The materials, methods, and examples presented herein are intended to be illustrative, and are not intended to limit the scope of the invention. All publications, including patent applications, patents, books, and other references mentioned herein are incorporated by reference in their entirety.

What is claimed is:

1. A compound having the Formula I: 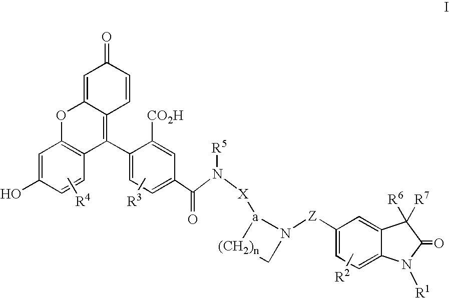
or a salt thereof; wherein:
R¹ is H, C₁-C₆ alkyl or C₇-C₂₄ aralkyl;
R², R³ and R⁴ are, each independently, H, halogen or C₁-C₆ alkyl;
R⁵ is H or C₁-C₆ alkyl;
n is 1, 2 or 3;
X is —(CH₂)ₘCONR^{5a}—(CH₂)_{q}—, —(CH₂)_{q}— or —(CH₂)ₘSO₂NR^{5a}(CH₂)_{q}—;
R^{5a} is H or C₁-C₆ alkyl;
m is 1, 2, 3, 4, 5 or 6;
q is 0, 1, 2 or 3;
R⁶ and R⁷ are each independently OH or —O—(C₁-C₆ alkyl);
or R⁶ and R⁷ taken together form ═O or —O—(CH₂), —O—, where r is 2 or 3; and
Z is CO or SO₂; and
a is a carbon atom.

2. A compound of claim 1 wherein the carbon atom designated “a” has the S configuration.

3. A compound of claim 2 wherein n is 2.

4. A compound of claim 2 wherein X is —CH₂—.

5. A compound of claim 2 wherein X is —(CH₂)ₘCONR^{5a}(CH₂)_{q}—.

6. A compound of claim 2 wherein X is —(CH₂)ₘCONR^{5a}(CH₂)_{q}—, m is 5 and q is 1.

7. A compound of claim 2 wherein X is —(CH₂)ₘCONR^{5a}(CH₂)_{q}—, m is 5, q is 1, and R^{5a} is hydrogen.

8. A compound of claim 2 wherein Z is SO₂.

9. A compound of claim 2 wherein Z is CO.

10. A compound of claim 2 wherein R², R³, R⁴, R⁵ and R^{5a} are each H.

11. A compound of claim 2 wherein R¹ is methyl.

12. A compound of claim 2 wherein R⁶ and R⁷ taken together form ═O.

13. A compound of claim 2 wherein:
n is 2;
X is —CH₂—;
Z is SO₂;
R², R³, R⁴ and R⁵ are each hydrogen;
R⁶ and R⁷ are taken together to form ═O; and
R¹ is methyl.

14. A compound of claim 2 wherein:
n is 2;
X is —(CH₂)ₘCONR^{5a}(CH₂)_{q}—, wherein m is 5, q is 1, and R^{5a} is hydrogen;
Z is SO₂;
R², R³, R⁴ and R⁵ are each hydrogen;
R⁶ and R⁷ are taken together to form ═O; and
R¹ is methyl.

15. A compound having the Formula IIa: 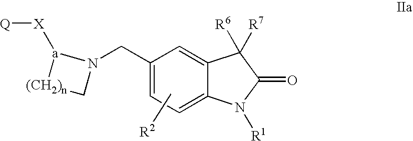
or a salt thereof; wherein:
R¹ is H, C₁-C₆ alkyl or C₇-C₂₄ aralkyl;
R² is H, halogen or C₁-C₆ alkyl;
n is 1, 2 or 3;
X is —(CH₂)_{q}—;
q is 0, 1, 2 or 3;
R⁶ and R⁷ are each independently OH or —O—(C₁-C₆ alkyl);
or R⁶ and R⁷ taken together can form ═O or —O—(CH₂)ᵣ—O—, where r is 2 or 3;
Z is CO or SO₂;
Q is N(R¹⁰)(R¹¹);
R¹⁰ and R¹¹ are each independently H or a protecting group;
or R¹⁰ and R¹¹ taken together with the nitrogen atom to which they are attached can form an N-phthalimidyl group; and
a is a carbon atom.

16. A compound of claim 15 wherein the carbon atom designated “a” has the S configuration.

17. A compound of claim 16 wherein R¹ is H.

18. A compound of claim 16 wherein R⁶ and R⁷ taken together form —O—(CH₂)ᵣ—O—, where r is 2 or 3.

19. A compound of claim 16 wherein n is 2.

20. A compound of claim 16 wherein X is —CH₂—.

21. A compound of claim 16 wherein R⁶ and R⁷ taken together form ═O.

22. A compound of claim 16 wherein R¹ and R² are each H; n is 2; X is —CH₂—; R⁶ and R⁷ taken together form ═O; and R¹⁰ and R¹¹ taken together with the nitrogen atom to which they are attached form an N-phthalimidyl group.

23. A compound of claim 16 wherein R¹ and R² are each H; n is 2; X is —CH₂—; R⁶ and R⁷ taken together form —O—(CH₂)₃—O—; and R¹⁰ and R¹¹ taken together with the nitrogen atom to which they are attached form an N-phthalimidyl group.

24. A compound of claim 16 wherein R¹ is methyl; R² is H; n is 2; X is —CH₂—; R⁶ and R⁷ taken together form —O—(CH₂)₃—O—; and R¹⁰ and R¹¹ taken together with the nitrogen atom to which they are attached form an N-phthalimidyl group.

25. A compound of claim 16 wherein R¹ is methyl; R² is H; n is 2; X is —CH₂—; R⁶ and R⁷ taken together form —O—(CH₂)₃—O—; and Q is NH₂.

26. A method for the preparation of a compound of Formula I: 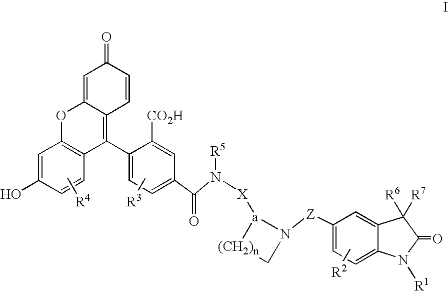
or a salt thereof; wherein:
R¹ is H, C₁-C₆ alkyl or C₇-C₂₄ aralkyl;
R², R³ and R⁴ are, each independently, H, halogen or C₁-C₆ alkyl;
R⁵ is H or C₁-C₆ alkyl;
n is 1, 2 or 3;
X is —(CH₂)ₘCONR^{5a}—(CH₂)_{q}—, —(CH₂)_{q}— or —(CH₂)ₘSO₂NR^{5a}—CH₂)_{q}—;
R^{5a} is H or C₁-C₆ alkyl;
m is 1, 2, 3, 4, 5 or 6;
q is 0, 1, 2 or 3;
R⁶ and R⁷ are each independently OH or —O—(C₁-C₆ alkyl);
or R⁶ and R⁷ taken together form ═O or —O—(CH₂), —O—, where r is 2 or 3;
Z is CO or SO₂; and
a is a carbon atom;
the method comprising:
a) providing a compound of Formula II: 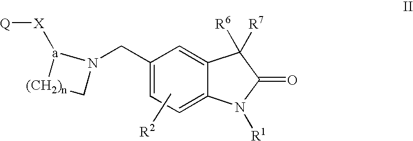
or a salt thereof, wherein:
X is —(CH₂)_{q}—;
R⁶ and R⁷ taken together form —O—(CH₂)ᵣ—O—, where r is 2 or 3; and
Q is NH₂; and
b) reacting the compound of Formula II with a compound of Formula III: 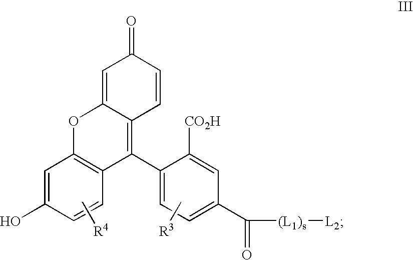
or a salt thereof, wherein:
L₁ is a linker group;
s is 0 or 1; and
L₂ is a leaving group;
for a time and under conditions effective to form the compound of Formula I.

27. The method of claim 26 wherein carbon atom designated “a” has the S configuration.

28. The method of claim 27 wherein L₁ has the formula —NR⁵—(CH₂)ₘCO—, —NR⁵—(CH₂)ₘSO₂ or —NH—(CH₂)ₘ—C(═O)—.

29. The method of claim 27 wherein L₂ has the formula:

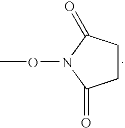

30. The method of claim 27 wherein L₁ has the formula —NH—(CH₂)ₘ—C(═O)—; and L₂ has the formula:

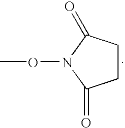

31. The method of claim 30 wherein m is 5.

32. The method of claim 27 wherein s is 0.

33. The method of claim 27 wherein s is 0 and L₂ has the formula:

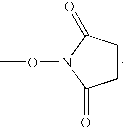

34. The method of claim 27 wherein:
n is 2;
X is —CH₂—;
Z is SO₂;
R², R³, R⁴ and R⁵ are each hydrogen;
R⁶ and R⁷ taken together form —O—(CH₂)₃—O—;
R¹ is methyl;
s is 0; and L₂ has the formula:

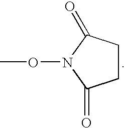

35. The method of claim 27 wherein:
n is 2;
X is —(CH₂)ₘCONR^{5a}(CH₂)_{q}, wherein m is 5, q is 1, and R^{5a} is hydrogen;
Z is SO₂;
R², R³, R⁴ and R⁵ are each hydrogen;
R⁶ and R⁷ taken together form —O—(CH₂)₃—O—;
R¹ is methyl;
s is 1;
L₁ has the formula —NH—(CH₂)ₘ—C(═O)—; and
L₂ has the formula:

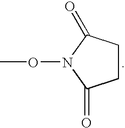

36. The method of claim 27 further comprising reacting the compound of Formula I wherein R⁶ and R⁷ taken together form —O—(CH₂)ᵣ—O—, where r is 2 or 3, with an acid to form a compound of Formula I wherein R⁶ and R⁷ taken together form ═O.

37. The method of claim 34 further comprising reacting the compound of Formula I wherein R⁶ and R⁷ taken together form —O—(CH₂)₃—O—, with an acid to form a compound of Formula I wherein R⁶ and R⁷ taken together form ═O.

38. The method of claim 35 further comprising reacting the compound of Formula I wherein R⁶ and R⁷ taken together form —O—(CH₂)₃—O—, with an acid to form a compound of Formula I wherein R⁶ and R⁷ taken together form ═O.

39. A method for the preparation of a compound of Formula II: 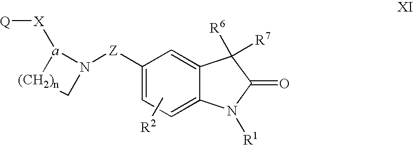
wherein:
R¹ is C₁-C₆ alkyl or C₇-C₂₄ aralkyl;
R² is H, halogen or C₁-C₆ alkyl;
n is 1, 2 or 3;
X is —(CH₂)_{q}—;
q is 0, 1, 2 or 3;
R⁶ and R⁷ taken together form —O—(CH₂)ᵣ—O—, where r is 2 or 3;
Z is CO or SO₂;
Q is NH₂; and
a is a carbon atom;
comprising:
a) providing a compound of Formula V: 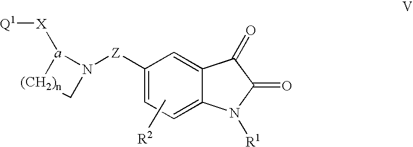
wherein:
R¹ is H;
Q¹ is N(R¹⁰)(R¹¹); and
R¹⁰ is a protecting group and R¹¹ is H;
or R¹⁰ and R¹¹ taken together with the nitrogen atom to which they are attached form an N-phthalimidyl group;
b) reacting the compound of Formula V with a reagent having the formula HO—(CH₂)ᵣ—OH where r is 2 or 3, under conditions effective to form a compound of Formula VI: 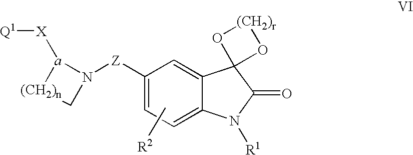
c) reacting the compound of Formula VI formed in step b) with an alkyl iodide or aralkyl iodide to form a compound of Formula VI wherein R¹ is alkyl or aralkyl; and
d) treating the compound of Formula VI wherein R¹ is alkyl or aralkyl formed in step c) with a reagent effective to remove:
i) the protecting group from R¹⁰; or
ii) the N-phthalimidyl group from N(R¹⁰)(R¹¹);
to form the compound of Formula II.

40. The method of claim 39 wherein carbon atom designated “a” has the S configuration.

41. The method of claim 40 wherein r is 3.

42. The method of claim 40 wherein n is 2.

43. The method of claim 40 wherein R¹⁰ and R¹¹ taken together with the nitrogen atom to which they are attached form an N-phthalimidyl group.

44. The method of claim 40 wherein R² is H.

45. The method of claim 40 wherein step c) comprises reacting the compound of Formula VI from step b) with an alkyl iodide to form a compound of Formula VI wherein R¹ is alkyl.

46. The method of claim 40 wherein step c) comprises reacting the compound of Formula VI from step b) with methyl iodide to form a compound of Formula VI wherein R¹ is methyl.

47. The method of claim 40 wherein r is 3; n is 2; R¹⁰ and R¹¹ taken together with the nitrogen atom to which they are attached form an N-phthalimidyl group; R² is H; and step c) comprises reacting the compound of Formula VI from step b) with methyl iodide to form a compound of Formula VI wherein R¹ is methyl.

48. A fluorescence polarization assay for determining whether a test compound binds to a caspase, the assay comprising:
(a) combining the test compound, a fluorescent probe, and a caspase in a solution;
(b) incubating the solution until equilibrium has been reached; and
(c) determining the difference in the amount of probe bound to the caspase in the presence and absence of the test compound;
wherein the probe is a compound of claim 1.

49. The method of claim 48 wherein the concentration of caspase in the solution is selected to provide a preselected value of fraction of probe bound to the caspase.

50. The method of claim 49 wherein the selecting of the concentration of caspase in the solution comprises:
i) incubating the probe with varying concentrations of caspase;
ii) measuring fluorescence polarization values for the varying concentrations of caspase;
iii) converting the measured fluorescence polarization values to values of fraction bound; and
iv) selecting a caspase concentration providing a desired value of fraction bound.

51. The method of claim 50 wherein step (iii) comprises converting the fluorescence polarization values to anisotropy values.

52. The method of claim 48 wherein step (c) comprises:
(c₁) determining the fluorescence polarization value of the fluorescent probe in the solution;
(c₂) determining the fraction of probe bound to the caspase from the fluorescence polarization value; and
(c₃) comparing the fraction of probe bound to the caspase to the fraction of probe bound to the caspase in the absence of the test compound.

53. The method of claim 52 wherein step (c₂) comprises converting the fluorescence polarization values to anisotropy values.

54. The method of claim 48 wherein step (a) comprises contemporaneously combining together the caspase, the probe, and the test compound.

55. The method of claim 48 wherein step (a) comprises:
(a₁) combining the caspase and the test compound together to form a mixture;
(a₂) waiting for a period of time; and
(a₃) adding the probe to the mixture.

56. The method of claim 48 wherein step (a) comprises:
(a₁) combining the caspase and the probe together to form a mixture;
(a₂) waiting for a period of time; and
(a₃) adding the test compound to the mixture.

57. The method of claim 48 further comprising performing the steps (a)-(c) a plurality of times, each with a different concentration of test compound.

58. The method of claim 57 further comprising determining a Ki value for the test compound.

59. A fluorescence polarization assay for screening a plurality of test compound for binding to a caspase, the assay comprising:
(a) providing a plurality of test solutions, each containing a test compound, a fluorescent probe, and a caspase;
(b) incubating the solutions until equilibrium has been reached; and
(c) determining the differences in the amounts of probe bound to the caspase in the presence and absence of the test compounds;
wherein the probe is a compound of claim 1.

60. The method of claim 59 wherein the concentration of caspase in the test solutions is selected to provide a preselected value of fraction of probe bound to the caspase.

61. The method of claim 60 wherein the selecting of the concentration of caspase in the test solutions comprises:
i) incubating the probe with varying concentrations of caspase;
ii) measuring fluorescence polarization values for the varying concentrations of caspase;
iii) converting the measured fluorescence polarization values to values of fraction bound; and
iv) selecting a caspase concentration providing a desired value of fraction of probe bound to the caspase.

62. The method of claim 61 wherein step (iii) comprises converting the fluorescence polarization values to anisotropy values.

63. The method of claim 59 wherein for each test solution, step (c) comprises:
(c₁) determining the fluorescence polarization value of the fluorescent probe in the solution;
(c₂) determining the fraction of probe bound to the caspase from the fluorescence polarization value; and
(C₃) comparing the fraction of probe bound to the caspase to the fraction of probe bound to the caspase in the absence of the test compound.

64. The method of claim 63 wherein for each test solution, step (c₂) comprises converting the fluorescence polarization values to anisotropy values.

65. The method of claim 59 wherein for each test solution, step (a) comprises contemporaneously combining together the caspase, the probe, and the test compound.

66. The method of claim 59 wherein for each test solution, step (a) comprises:
(a₁) combining the caspase and the test compound together to form a mixture;
(a₂) waiting for a period of time; and
(a₃) adding the probe to the mixture.

67. The method of claim 59 wherein for each test solution, step (a) comprises:
(a₁) combining the caspase and the probe together to form a mixture;
(a₂) waiting for a period of time; and
(a₃) adding the test compound to the mixture.

68. The method of claim 59 further comprising performing the steps (a)-(c) a plurality of times, each with a different concentration of test compound.

69. The method of claim 68 further comprising determining a Ki value for the test compound.

70. A product of the method of claim 27.

71. A product of the method of claim 27 wherein:
n is 2;
X is —CH₂—;
Z is SO₂;
R², R³, R⁴ and R⁵ are each hydrogen;
R⁶ and R⁷ are taken together to form ═O; and
R¹ is methyl.

72. A product of the method of claim 27 wherein:
n is 2;
X is —(CH₂)ₘCONR^{5a}(CH₂)_{q}, wherein m is 5, q is 1, and R^{5a} is hydrogen;
Z is SO₂;
R², R³, R⁴ and R⁵ are each hydrogen;
R⁶ and R⁷ are taken together to form ═O; and
R¹ is methyl.

73. A product of the method of claim 39.

74. A product of the method of claim 40.

75. A product of the method of claim 40 wherein:
r is 3;
n is 2;
R¹⁰ and R¹¹ taken together with the nitrogen atom to which they are attached form an N-phthalimidyl group;
R² is H; and step c) comprises reacting the compound of Formula VI from step b) with methyl iodide to form a compound of Formula VI wherein R¹ is methyl.
